(19) United States
(12) Patent Application Publication
(10) Pub. No.: US 2023/0405249 A1
(43) Pub. Date: Dec. 21, 2023

(54) DEVICE AND SYSTEM FOR RESPIRATORY THERAPY

(71) Applicant: Loewenstein Medical Technology S.A., Luxembourg (LU)

(72) Inventors: Jan SCHATTNER, Pinneberg (DE); Christof GOEBEL, Hamburg (DE)

(21) Appl. No.: 18/329,713

(22) Filed: Jun. 6, 2023

(30) Foreign Application Priority Data
Jun. 21, 2022 (DE) 102022115391.6

(51) Int. Cl. A61M 16/00

(52) U.S. Cl. CPC A61M 16/022; A61M 16/0003; A61M 2205/502

(57) ABSTRACT

A device for respiratory therapy of a patient comprises a respiratory gas source for specifying different respiratory gas parameters, comprising at least one control unit, and comprising a signal unit for outputting at least one signal. The at least one signal is used for signaling changing respiratory gas parameters and is sensorially perceptible by the patient.

### CROSS-REFERENCE TO RELATED APPLICATIONS

The present application claims priority under 35 U.S.C. § 119 of German Patent Application No.102022115391.6, filed Jun. 21, 2022, the entire disclosure of which is expressly incorporated by reference herein.

### BACKGROUND OF THE INVENTION

### 1. Field of the Invention

The invention relates to respiratory therapy in patients having impaired or weak coughing, for whom coughing up secretion in the airways is more difficult or is not possible (insufficient coughing)—for example, based on a lack of muscular force.

### 2. Discussion of Background Information

Airway secretion has thixotropic properties, that is to say shear due to gas flowing past in the airway initially does not cause liquefying or movement of secretion below a certain degree. The viscosity of the secretion only changes when the shear exceeds a threshold value and it is set into movement. Reaching this degree of shear stress by the (expiratory) gas flowing past requires a certain flow (Peak Flow, Peak Cough Flow) during the coughing. This is in turn linked to certain conditions and requires a minimum amount of muscular force without machine assistance.

The procedure of sufficient coughing can be divided into 3 phases. The first phase of the deepest possible insufflation and filling of the lung (with open glottis) is followed by closing the glottis with immediately following compression and pressure buildup with closed glottis and finally the cough with glottis open again, during which the greatest possible Peak Flow (Peak Cough Flow, PCF) is to be generated.

A respiratory therapy can be necessary for patients if a cough insufficiency or a deficient muscular force is present, to assist the removal of secretion from the airways. Such a therapy can comprise a mechanical (pneumatic) insufflation/exsufflation by a respiratory therapy device. In the exsufflation phase, the energy for generating the airflow during the cough is applied here by the respiratory therapy device or the natural cough is at least assisted.

For a sufficient—mechanically assisted—cough, in addition to the application of the required energy by the respiratory therapy device, a good synchronization of the various described phases between respiratory therapy device and patient is also included. For example, if the glottis closure by the patient before the cough were to take place too late, after the therapy device has already switched into the exsufflation phase, expiratory flow would be generated without coughing (without the required peak flow): the coughing would then not be effective. On the other hand, if the glottis closure by the patient were to take place early—thus before completion of the insufflation phase—the lung would possibly not yet be adequately filled and the following coughing would again not be optimum and possibly also inadequate. In addition, if the device-side mechanical assistance and the muscle pressure buildup by the patient do not coincide in time, the individual best-possible coughing and peak cough flow will also not be achieved. For these reasons, the synchronization between the patient and the respiratory therapy device is of great importance for a sufficient mechanical cough therapy.

Insufflation and exsufflation times can be produced by means of settable time specifications, wherein the setting physician has the responsibility of selecting suitable times and setting them on the respiratory therapy device.

The selection of the insufflation time can alternatively also be determined automatically and on the basis of respiratory measured values. According to this method, patient-side changes such as the degree of the (increasing) lung filling and/or the patency of the upper airways are reacted to on the basis of the measured values and of detector signals. Metrologically determinable limiting states relevant according to the invention can be, for example, an at least substantially filled lung or a substantial or complete glottis closure (at the end of the insufflation phase).

According to the prior art, optical or acoustic signals can be used to optimize this synchronization, which notify the patient of a change between these phases. These signals generated on the device side can take place on the basis of the times stored on the device side or settable on the user side via a user interface.

In a clinical application, a physician can further improve the mechanically assisted cough by manual compression of the stomach area of the patient during the exsufflation. This compression has to take place here at the correct moment, namely synchronized with the exhalation or the cough of the patient.

EP 2 651 477 B 1, the entire disclosure of which is incorporated by reference herein, discloses a respiratory therapy device which indicates to the physician the correct moment for the assisting compression via a tone signal or via an image signal or a light signal.

The disadvantages are that the surroundings can be disturbed by acoustic signals or that the perception of acoustic signals can be made more difficult by loud surroundings.

Light (direct sunlight) incident on the therapy device can have an interfering effect with respect to the perception of optical signals from the surroundings and impair the perception of these provided optical signals. One significant disadvantage, however, is that a physician or helper is required.

Furthermore, the patient has to use his acoustic or visual sense according to the prior art while his concentration and focusing is preferably directed to the intense mechanical cough maneuver—thus inward as it were.

A further aspect in typical application scenarios is that the use of medical devices is in any case often accompanied by acoustic stimuli—perceived as annoying—for example by the intrinsic noises of the devices or also by—sometimes unnecessary—alarms of the devices.

In view of the foregoing, it would be advantageous to have available a device for respiratory therapy which overcomes the deficiencies.

One essential advantage of the present invention may be that the patient receives the signaling of changing respiratory gas parameters on the airway and thus in a very direct and well perceptible manner and is thus capable of achieving optimum synchronization of her own cough with the respiratory therapy device.

A further advantage of the present invention may be that the signals according to the invention do not address other senses such as the eye or ear of the patient she he has directed all of her attention to the procedure of lung filling and preparing for the cough.

One special advantage of the present invention may be that that she can perceive the signals according to the invention with her respiratory system.

According to the invention, the respiratory system comprises at least the lung, the upper and lower airways, and also areas of the face which can be in contact with respiratory gas.

A further advantage of the present invention may be that the signals according to the invention are particularly suitable for visually-impaired and/or hearing-impaired persons, who can perceive acoustic or visual stimuli not at all or only to a limited extent.

### SUMMARY OF THE INVENTION

The invention provides a device for respiratory therapy of a patient comprising a respiratory gas source for specifying different respiratory gas parameters, comprising at least one control unit, and comprising a signal unit for outputting at least one signal. The device is characterized in that the signal is used for signaling changing respiratory gas parameters and is sensorially perceptible by the patient.

In some embodiments, the device is characterized in that the signal unit emits the signal to the patient before the change of a respiratory gas parameter.

In some embodiments, the device is characterized in that the signal is transmitted to the airway of the patient pneumatically—thus via the air path (device, hose system; patient interface).

In some embodiments, the device is characterized in that the signal includes a modulation of the specified respiratory gas with respect to pressure and/or flow and/or volume.

In some embodiments, the device is characterized in that the signal is preferably sensorially perceptible by the patient via his mechanoreceptors or cold receptors.

In some embodiments, the device is characterized in that the signal is preferably sensorially perceptible by the patient via his smell receptors or taste receptors.

In some embodiments, the device is characterized in that the signal unit is configured and designed to generate those signals which are perceptible by the patient via his mechanoreceptors or cold receptors.

In some embodiments, the device is characterized in that the signal unit is configured and designed to generate those signals which are perceptible by the patient via his mechanoreceptors and acoustically.

In some embodiments, the device is characterized in that the signal is not visually perceptible and is not a light signal.

In some embodiments, the device is characterized in that the signal unit is designed to generate pneumatic signals and includes, for example, a valve and/or a fan.

In some embodiments, the device is characterized in that the respiratory gas source is a fan which is also used as a signal unit.

In some embodiments, the device is characterized in that the signal is generated by the respiratory gas source.

In some embodiments, the device is characterized in that the signal unit is controlled by the control unit.

In some embodiments, the device is characterized in that the changing respiratory gas parameter is the respiratory gas pressure provided by the device.

In some embodiments, the device is characterized in that the respiratory gas parameter is changed upon switching from insufflation to coughing phase (expiration and/or exsufflation).

In some embodiments, the device is characterized in that the changing respiratory gas parameter is a positive respiratory gas pressure, which is dissipated following the insufflation phase for the coughing phase (expiration) or is switched to negative pressure (exsufflation).

In some embodiments, the device is characterized in that the device carries out an insufflation with overlaid oscillation, wherein the signal is an oscillation which is changed before switching to the coughing phase (exsufflation and/or expiration).

In some embodiments, the device is characterized in that the signal consists in the oscillation being switched off before switching to the coughing phase.

In some embodiments, the device is characterized in that the signal consists in an existing oscillation being amplified.

In some embodiments, the device is characterized in that the device carries out an insufflation without oscillation, wherein the signal is a specific modulation of the respiratory gas with respect to pressure and/or flow and/or volume.

In some embodiments, the device is characterized in that the signal includes a modulation of pressure and/or flow (pneumatic signal).

In some embodiments, the device is characterized in that the signal is a modulated-on oscillation having a fixed or changing frequency and/or amplitude.

In some embodiments, the device is characterized in that the oscillation has a frequency from about 1 Hz to about 40 Hz.

In some embodiments, the device is characterized in that the oscillation preferably has a frequency from about 2 Hz to about 30 Hz.

In some embodiments, the device is characterized in that the oscillation in turn preferably has a frequency from about 3 Hz to about 15 Hz.

In some embodiments, the device is characterized in that the amplitude of the oscillation (peak to peak) is from about 0.2 hPa to about 50 hPa.

In some embodiments, the device is characterized in that the amplitude of the oscillation (peak to peak) is preferably from about 0.5 hPa to about 30 hPa.

In some embodiments, the device is characterized in that the amplitude of the oscillation (peak to peak) is in turn preferably from about 1 hPa to about 20 hPa.

In some embodiments, the device is characterized in that the frequency and/or amplitude change by rising or change by falling.

In some embodiments, the device is characterized in that the frequency and/or amplitude change dynamically.

In some embodiments, the device is characterized in that the signal is a brief positive or negative pressure and/or flow pulse on the air column.

In some embodiments, the device is characterized in that the device briefly raises a respiratory gas pressure starting from a specific pressure toward the end of the insufflation phase and then lowers it back to the prior level (plateau).

In some embodiments, the device is characterized in that the pressure is raised at least once to the then highest pressure toward the end of the insufflation phase.

In some embodiments, the device is characterized in that the pressure briefly sinks and then rises again to the prior level (plateau).

In some embodiments, the device is characterized in that the pressure drops again slightly toward the end of the insufflation phase.

In some embodiments, the device is characterized in that a (temporary) pressure adjustment is in the range of from about 0.2 hPa to about 8 hPa.

In some embodiments, the device is characterized in that a (temporary) pressure adjustment is in the range of from about 0.5 hPa to about 5 hPa.

In some embodiments, the device is characterized in that the signal changes between the beginning of the signal and the moment of switching.

In some embodiments, the device is characterized in that the device moreover includes a generator for generating a detector signal and a sensor for determining a change of the detector signal, wherein the detector signal is suitable for detecting changes of the patency of the airways (closure of the glottis) and/or an at least advanced or complete filling of the lung and the sensor determines this change of the detector signal.

In some embodiments, the device is characterized in that automatic switching to the coughing phase takes place when, based on the detector signal, a change of the patency of the airways (closure of the glottis) or an at least advanced or complete filling of the lung is determined.

In some embodiments, the device is characterized in that the detector signal is an oscillatory stimulation and/or response and in that a change of the airway impedance is determined via a change of the detector signal.

In some embodiments, the device is characterized in that upon sudden change (rise) of the airway impedance, a closure of the glottis is metrologically concluded.

In some embodiments, the device is characterized in that the automatic switching is based on an oscillatory pressure, flow, and/or volume signal.

In some embodiments, the device is characterized in that the switching also takes place before expiration of a stored or set period of time if the glottis closure is established metrologically by a detector signal.

In some embodiments, the device is characterized in that the preset period of time then becomes a maximum duration, which can also be shortened based on a detector signal.

In some embodiments, the device is characterized in that it is possible to distinguish between open and closed glottis based on the oscillatory pressure signal.

In some embodiments, the device is characterized in that it is possible to conclude the degree of filling of the lung based on the oscillatory pressure signal.

In some embodiments, the device is characterized in that the oscillation takes place during the insufflation, wherein the oscillation is simultaneously used as the basis for the detector signal and switching takes place when, based on the detector signal, a closure of the glottis and/or an at least substantially completely filled lung can be concluded before expiration of the set insufflation time.

In some embodiments, the device is characterized in that the oscillation as the basis for the detector signal for the glottis closure is added in the course of the insufflation, wherein the insufflation is ended by a detected glottis closure or with expiration of the set maximum insufflation time.

In some embodiments, the device is characterized in that the pneumatic signal is generated for a fixed and/or settable period of time.

In some embodiments, the device is characterized in that the pneumatic signal is generated from a fixed and/or settable time before switching to the coughing phase.

In some embodiments, the device is characterized in that the period of time of generating the signal is from about 0.1 seconds to about 2 seconds.

In some embodiments, the device is characterized in that the period of time of generating the signal is from about 0.5 seconds to about 1.5 seconds.

In some embodiments, the device is characterized in that the period of time of generating the signal is from about 0.8 seconds to about 1.2 seconds.

In some embodiments, the device is characterized in that the time before switching from insufflation to exsufflation and/or expiration, at which the generation of the signal is begun, is from about 0.1 seconds to about 2 seconds.

In some embodiments, the device is characterized in that the time before switching from insufflation to exsufflation and/or expiration, at which the generation of the signal is begun, is from about 0.5 seconds to about 1.5 seconds.

In some embodiments, the device is characterized in that the time before switching from insufflation to exsufflation and/or expiration, at which the generation of the signal is begun, is from about 0.8 seconds to about 1.2 seconds.

In some embodiments, the device is characterized in that the pneumatic signal is generated over a settable time before switching to exsufflation.

In some embodiments, the device is characterized in that the pneumatic signal is generated on the basis of pressure and flow values.

In some embodiments, the device is characterized in that pressure and/or flow are measured during a maneuver.

In some embodiments, the device is characterized in that the pneumatic signal is generated on the basis of the detector signal during the insufflation phase.

In some embodiments, the device is characterized in that, when the flow is close to about 0 /lmin (thus the patient lung is fully filled), the pneumatic signal is generated.

In some embodiments, the device is characterized in that the signal is generated by a valve integrated in the device.

In some embodiments, the device is characterized in that the signal is generated by an actuator integrated in the device, which operates according to the displacer principle.

In some embodiments, the device is characterized in that the actuator operating according to the displacer principle is a piston.

In some embodiments, the device is characterized in that an oscillation is generated by means of an oscillatory pressure/flow pump.

In some embodiments, the device is characterized in that the oscillation and/or the pressure jump is generated by means of a valve integrated in the device.

In some embodiments, the device is characterized in that the flow and/or pressure reduction of the oscillation is generated by variable leaks.

In some embodiments, the device is characterized in that the flow and/or pressure reduction of the oscillation is generated by reciprocal connection of the airways to the pressure and suction side of at least one flow source.

In some embodiments, the device is characterized in that the flow or pressure source is a fan.

In some embodiments, the device is characterized in that the oscillation and/or the pressure jump is generated by one or more volume shifts.

In some embodiments, the device is characterized in that the signal is generated by variation and/or adjustment of the speed of the at least one fan integrated in the device.

In some embodiments, the device is characterized in that, toward the end of the insufflation phase, the device temporarily keeps the pressure during a plateau time at a level and the signal unit initiates the output of a signal before switching to a lower pressure, so that the patient can close the glottis before the pressure is dissipated and the cough begins.

The invention further provides a system for respiratory therapy of a patient comprising the device according to the invention, wherein the system moreover comprises a patient interface and a respiratory gas hose.

It is to be noted that the features listed individually in the claims and the description can be combined with one another in any technically reasonable manner and disclose further embodiments of the invention. The description additionally characterizes and specifies the invention especially in conjunction with the figures.

It is furthermore to be noted that an “and/or” conjunction used herein, which is between two features and links them to one another, is always to be interpreted to mean that in a first embodiment of the subject matter according to the invention only the first feature can be present, in a second embodiment only the second feature can be present, and in a third embodiment both the first and the second feature can be present.

The device for respiratory therapy of a patient comprises at least one respiratory gas source for specifying different respiratory gas parameters, at least one control unit, and at least one signal unit for outputting at least one signal. The signal is sensorially perceptible by the patient and is used for signaling changing respiratory gas parameters. Changing respiratory gas parameters in the context of the invention are in particular changes of the respiratory gas parameters caused by the device. In some embodiments, the signal is transmitted at least pneumatically, thus via the air path to the airway of the patient. For example, the air path comprises at least the device, a hose system coupled to the device, and a patient interface coupled to the hose system. In some embodiments, the signal consists in a modulation of the pressure and/or flow to the patient.

In some embodiments, the signal is used for signaling changing respiratory gas parameters in the form of the upcoming switching from mechanical insufflation to expiration and/or exsufflation in the course of a cough therapy and/or cough assistance. The expiration and/or exsufflation represents a coughing phase. The signal thus signals switching to the coughing phase. In some embodiments, during a cough therapy, the insufflation is followed by a sudden switching off of the positive insufflation pressure, due to which an expiration is forced and possibly also a cough of the patient is effectuated. A switch thus takes place from insufflation to exsufflation and/or expiration. In some embodiments, the insufflation is followed by an exsufflation which generates a higher pressure difference in relation to the insufflation and thus also effectuates higher energy for the cough reaction. In some embodiments, the signal indicates to the patient that he can or should manually switch to expiration and/or exsufflation.

In some embodiments, the signal is independent of whether the coughing phase begins due to switching, following an insufflation, to an expiration or exsufflation. Switching to the coughing phase in some embodiments means in particular switching to expiration and/or exsufflation.

In some embodiments, it can be provided that the insufflation is carried out with a superimposed oscillation, wherein the signal consists in the oscillation being changed and/or switched off before switching from insufflation to an expiration and/or exsufflation. A change of the oscillation can take place, for example, via strengthening or weakening of the oscillation. For example, the amplitude and/or the frequency of the oscillation is increased or decreased.

If the insufflation is carried out without superimposed oscillation, it is provided that the signal consists in carrying out a specific modulation before switching to the coughing phase with exsufflation or expiration.

In some embodiments, it is provided that the signal consists in a modulation of pressure and/or flow, for example as a pneumatic signal. For example, it can be provided that the signal is an oscillation of the pressure and/or the flow. The oscillation has in this case, for example, a frequency from about 1 Hz to about 40 Hz, preferably from about 2 Hz to about 30 Hz, more preferably from about 3 Hz to about 15 Hz. The peak to peak amplitude of the oscillation is, for example, from about 0.2 hPa to about 50 hPa, for example from about 0.5 hPa to about 30 hPa. In some embodiments, the amplitude is from about 1 hPa to about 20 hPa.

Alternatively or additionally, the signal is a short positive or negative pressure and/or flow pulse on the air column, for example in the pressure plateau phase, wherein the pulse is to be understood relative to the plateau pressure and/or flow. For example, the pressure rises briefly for signaling and then sinks back to the prior level, for example the plateau pressure. In some embodiments, it can be provided here that immediately after the sinking to the plateau pressure, the switching to exsufflation and/or expiration takes place. Alternatively, it can be provided that after the sinking to the plateau pressure, the plateau pressure is maintained for a short time before the switching takes place. In some embodiments, it is alternatively or additionally provided that for the signaling the pressure rises at the end of the plateau phase to a then highest pressure during the insufflation and the switching then takes place at the highest pressure. It can be provided that the switching takes place immediately after reaching the then highest pressure. Alternatively, it can be provided that the then highest pressure is maintained for a specific period of time before the switching takes place.

In some embodiments, it is provided that the signal consists in the pressure briefly sinking during the plateau phase and then rising back to the plateau level before the switching takes place. It can be provided here that immediately after the rise back to the plateau level, the switching takes place or the plateau level is still maintained for some time before the switching takes place. In some embodiments, it can be provided that the pressure is reduced for signaling before the switching and the switching takes place without rising again.

The level of the (temporary) pressure adjustment—increase or decrease—is from about 0.2 hPa to about 8 hPa, preferably from about 0.5 hPa to about 5 hPa.

In some embodiments, the signal can also change between the beginning of the signal and the switching. For example, the pressure adjustment can increase during the signal, thus extend rising and/or falling.

In some embodiments, the device is configured to carry out automatic switching to the coughing phase, thus expiration and/or exsufflation. The automatic switching can be based, for example, on an oscillatory pressure, flow, and/or volume signal. For example, it can be provided that an oscillation of the flow and/or the volume is generated as a stimulation by the device and the reaction of the airways to this oscillation, for example in the form of a pressure signal, is measured as the response. In some embodiments, it can be provided that in addition to the response, a possible phase shift between stimulation and response is also detected and/or evaluated.

In some embodiments, it can be provided that the signal is maintained over the period of time until automatic switching is triggered on the basis of the response and/or another detector signal. In some embodiments, a differentiation between a filled lung with open and closed glottis can also take place via the response of the airways. For example, a change of the airway impedance can be measured from the response. The status of the glottis (for example open/closed, possibly also partially closed), for example, can then be determined from the airway impedance. Upon a sudden rise of the airway impedance, for example, a closure of the glottis can be concluded, for example. If the glottis closure is established metrologically, for example, it can be provided that the switching from insufflation to expiration or exsufflation takes place. In some embodiments, a time is saved and/or set over which the insufflation is to take place or at least is to take place. If a glottis closure is established, the switching can also take place before expiration of the set or stored time. The set or stored time can thus, for example, also be a maximum time of the insufflation.

In some embodiments, the device comprises a unit (generator) for generating a detector signal and one or more sensors for determining changes of the detector signal. The detector signal is suitable, for example, for detecting the patency and/or the change of the patency of the airways of the patient. A reduced patency of the airways can indicate, for example, a closure of the glottis. In some embodiments, a closure of the glottis is taken as an indication that the patient has inhaled deeply enough and switching from insufflation to exsufflation or expiration can take place. Alternatively or additionally, the detector signal can be suitable for detecting the fill level and/or changes of the fill level of the lung of the patient.

In some embodiments, the detector signal is a flow, volume, and/or pressure signal. In some embodiments, the detector signal is generated by an oscillation of the pressure and/or flow. It can be provided, for example, that the unit for generating the detector signal is an oscillation valve.

In some embodiments, it can be provided that a superimposed oscillation takes place during the entire insufflation. This oscillation can be used here to determine, via a stimulation, a response of the airways of the patient, for example in the form of an oscillatory pressure signal, for example, to establish the closure of the glottis. For example, switching then takes place when a (possibly premature) closure of the glottis takes place.

In some embodiments, the oscillation is added as a signal to the signaling of the switching. It can be provided, for example, that a time is set or stored over which the insufflation is to take place before the switching, wherein the signal signals, for example, an expiration of this time and the upcoming switching. If the signaling takes place via an oscillation, for example of the pressure, this oscillation can be used to determine an oscillatory pressure signal. Since automatic switching takes place when a closure of the glottis is established, the stored or set time thus becomes a maximum time. In some embodiments, a minimum time can also be set or stored here over which period of time the insufflation is at least to take place.

In some embodiments, it is alternatively or additionally provided that the pneumatic signal is generated for a fixed period of time before switching to the exsufflation or the expiration. The patient can prepare for switching in accordance with the signal. In some embodiments, the fixed period of time is from about 0.1 seconds to about 2 seconds, preferably from about 0.5 seconds to about 1.5 seconds. In some embodiments, the fixed period of time before switching is preferably from about 0.8 seconds to about 1.2 seconds. In some embodiments, the time is additionally or alternatively settable from about 0 seconds to about 2 seconds.

In some embodiments, it can be provided that the intensity of the signal gradually becomes less after a number of therapies. For example, it can be provided that the patient becomes acclimated to the switching point in time in this way, so that the patient is less and less dependent on the signal and recognizes or can predict the upcoming switching more by himself. Such an attenuation of the signal is, for example, usable especially in conjunction with a fixed and/or settable period of time of the signal generation before switching and a fixed insufflation time. In some embodiments, it can also be provided that the synchronization between patient and device or therapy is evaluated and in the event of an increasing deviation—after attenuation of the signal—the signal is amplified again.

Alternatively or additionally, it is provided in some embodiments that the signal is generated on the basis of pressure and/or flow values. For example, the pressure and/or the flow are measured during insufflation. At specific pressures and/or flows it can then be provided that the signal is generated to signal switching from insufflation to expiration or exsufflation. For example, it can be provided that during the ventilation, the pressure and/or flow is evaluated as to whether the patient has inhaled deeply enough or insufflation is substantially completed, so that the switching takes place or can take place soon. For example, it can be provided that the signal is generated or output when the flow approaches 0 /lmin, the lung of the patient is thus fully filled. In some embodiments, it is provided that the device is configured to predict on the basis of the flow and/or pressure values when insufflation will be completed and switching takes place, so that from a fixed and/or settable time before the switching, the signal is generated for a fixed and/or settable period of time.

To generate the signal, it can be provided, for example, that the signal, for example in the form of a pressure adjustment and/or oscillation, is generated by means of a valve integrated in the device. A flow and/or pressure reduction, for example to generate an oscillation, can be generated here, for example, by variable leaks. In some embodiments, an oscillation and/or flow or pressure adjustment can take place by reciprocal connection of the respiratory gas path to the patient to the pressure and suction side of the pressure or flow source. For example, it can be provided that the pressure or flow is a fan.

In some embodiments, the signal is generated in the form of an oscillation and/or pressure adjustment via volume shifts. For example, the signal can be generated by a variation and/or adjustment of the speed of a fan, for example of the pressure or flow source. Alternatively, the volume shifts can also be generated via a displacement principle, for example by a piston. In some embodiments, an oscillation is generated via an oscillatory pressure and/or flow pump.

In some embodiments, the pneumatic signal is accompanied by an acoustic signal. In some embodiments, the acoustic signal arises due to the generation of the pneumatic signal. In some embodiments, it can be provided that the pneumatic signal is assisted by an additionally generated acoustic signal. Alternatively or additionally, it can be provided that the acoustic signal arising due to the generation of the pneumatic signal is additionally amplified.

### BRIEF DESCRIPTION OF THE DRAWINGS

The invention is illustrated in more detail on the basis of exemplary embodiments shown in the following drawings. In the drawings,

FIG. 1 schematically shows an exemplary embodiment of a device of the invention for respiratory therapy of a patient.

FIG. 2 schematically shows a further exemplary embodiment of a device of the invention for respiratory therapy of a patient.

FIG. 3 schematically shows a further exemplary embodiment of a device of the invention for respiratory therapy of a patient.

FIG. 4 schematically shows two exemplary pressure curves a) and b) of an insufflation and exsufflation or expiration.

FIG. 5 schematically shows another exemplary pressure curve of an insufflation and exsufflation or expiration.

FIG. 6 schematically shows another exemplary pressure curve of an insufflation and exsufflation or expiration.

FIG. 7 schematically shows another exemplary pressure curve of an insufflation and exsufflation or expiration.

FIG. 8 schematically shows another exemplary pressure curve of an insufflation and exsufflation or expiration.

FIG. 9 schematically shows another exemplary pressure curve of an insufflation and exsufflation or expiration.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

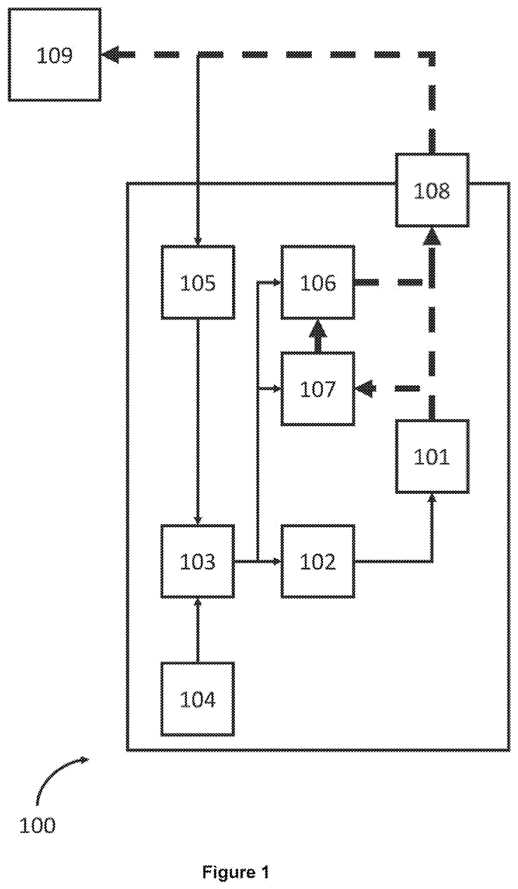

FIG. 1 schematically shows an exemplary embodiment of a device 100 for respiratory therapy of a patient 109. The device 100 comprises, for example, a flow or pressure source in the form of a fan 101, for example, driven by a controllable motor unit 102. At least one valve 107 and a signal unit 106 for generating a signal 204 for signaling changing respiratory gas parameters, for example, are pneumatically connected to the fan 101. The signal unit 106 is, for example, at the same time also an oscillation valve 110 for generating an oscillation of the pressure and/or flow. A pneumatic connection to the patient 109 is established via a hose system and patient interface (not shown) via the device outlet 108. Pressure and/or flow values are recorded, for example, via sensors 105.

At least the control of the motor unit 102 for driving the fan 101 and the control of the valve 107 and the signal unit 106 is implemented via the control unit 103.

An exemplary respiratory therapy comprises an insufflation 201 of the patient 109, wherein the overpressure of insufflation 201 is stopped suddenly or immediately (<500 ms) by switching 202. A (forced) expiration or an exsufflation 203 of the patient 109 follows thereon, also referred to as a coughing phase in the scope of the invention. The exemplary device 100 is configured so that an insufflation 201 and an exsufflation 203 are possible using the fan 101. During exsufflation 203, a negative pressure (in relation to the ambient pressure) is generated. Switching 202 from insufflation 201 to exsufflation 203 is possible, for example, by the valve 107. The suction side of the fan 101 is switched from ambient air to respiratory gas path by switching the valve 107. During insufflation 201, the fan 101 sucks in the ambient air and delivers this as respiratory gas in the direction of the patient 109. After switching, the suction side of the fan 101 is connected to the respiratory gas path; the fan thus sucks in on the side of the patient 109 and delivers in the direction of ambient air. A negative pressure on the side of the patient thus arises during an exsufflation 203. In some embodiments, it can be provided that the device 100 is additionally or alternatively configured so that, during the exemplary respiratory therapy, the insufflation 201 is not followed by exsufflation 203, rather the provision of an overpressure ends abruptly after insufflation 209 and thus an expiration and ideally a cough of the patient 109 is forced.

In some embodiments, it can be provided that switching from insufflation 201 to an expiration 203a is possible without switching the valve 107. For example, for this purpose the speed of the fan 101 is suddenly reduced to generate a pressure drop.

The change of respiratory gas parameters by switching 202 from insufflation 201 to expiration 203a and/or exsufflation 203 is signaled or announced according to the invention by a signal 204. In this case, this is preferably a pneumatic signal 204, which is generated in the exemplary device 100 via the signal unit 106. For example, the signal unit is in the form of an oscillation valve, which generates an oscillation of the insufflation pressure and/or flow, wherein the oscillation is at least used as the signal 204. In some embodiments, insufflation 201 is overlaid with an oscillation apart from the signal 204, generated by the oscillation valve 110. A signal 204 can be generated, for example, by stopping the oscillation or modulating the oscillation, for example, the amplitude and/or frequency change.

To generate the signal 204, for this purpose the signal unit 106 is activated accordingly by the control unit 103 and activated using corresponding control signals, for example, to implement specifications on the beginning, end, amplitude, and/or frequency of the signal 204 in the form of an oscillation.

If the signal 204 consists in an oscillation, the oscillation thus has a frequency from about 1 Hz to about 40 Hz, preferably from about 2 Hz to about 30 Hz. In some embodiments, the frequency of the oscillation is preferably from about 3 Hz to about 15 Hz. The amplitude of the oscillation which is used as the signal 204 is, for example, from about 0.2 hPa to about 50 hPa, preferably from about 0.5 hPa to about 30 hPa. In some embodiments, the amplitude of the oscillation of the signal 204 is from about 1 hPa to about 20 hPa. The amplitude designates the distance of the two peaks from one another.

Various respiratory gas parameters can be detected via the flow and/or pressure sensors 105. For example, it can be provided that it is determined via the oscillation during insufflation—both as the signal 204 and also apart from the signal 204—whether and/or when switching 202 is to take place. It can be provided that a minimum and/or maximum time is set and/or specified for insufflation. If the minimum insufflation time is reached, for example, the signal can be generated. The switching can then take place after a set or fixed period of time for which the signal 204 is generated. Alternatively or additionally, it can be provided that the switching takes place when it is established via the oscillation that switching is supposed to take place. In some embodiments, the settable or fixed period of time for the signal 204 becomes a maximum period of time, the switching thus takes place at the latest after expiration of the signal time. In some embodiments, alternatively or additionally, a maximum insufflation time is provided, after the expiration of which the switching 202 takes place independently of the oscillation measurements. For example, the maximum insufflation time can correspond to a minimum insufflation time plus a signal time. In some embodiments, in particular if no minimum insufflation time is provided, it can be provided that the signal 204 is generated in accordance with the duration of the signal 204 before the expiration of the maximum insufflation time.

It can alternatively or additionally be established via the sensors 105 whether the lung of the patient 109 is fully filled and switching 202 from insufflation 201 to expiration 203a or exsufflation 203 is to take place. For example, an approach of the flow to 0 /lmin can indicate a fully filled lung, so that the control unit 103 activates the signal unit 106 such that the signal 204 is generated to signal changing respiratory gas parameters—the switching from insufflation to exsufflation 203 or expiration 203a here.

Inputs of data, information, and/or parameters, for example, are possible via the user interface 104. Inter alia, parameters for the signal 204 can be input. For example, time before switching, period of time, frequency, amplitude, and/or intensity of the signal 204 can be set. The time is, for example, a period of time of the signal 204 and/or from when before the switching 202 from insufflation 201 to exsufflation 203 or expiration the signal 204 is generated. For example, it can be fixed and/or settable that the signal is generated over a period of time of 0.1 to 2 seconds. The settable and/or fixed period of time is preferably 0.5 seconds to 1.5 seconds. In some embodiments, the settable and/or fixed period of time is preferably 0.8 seconds to 1.2 seconds.

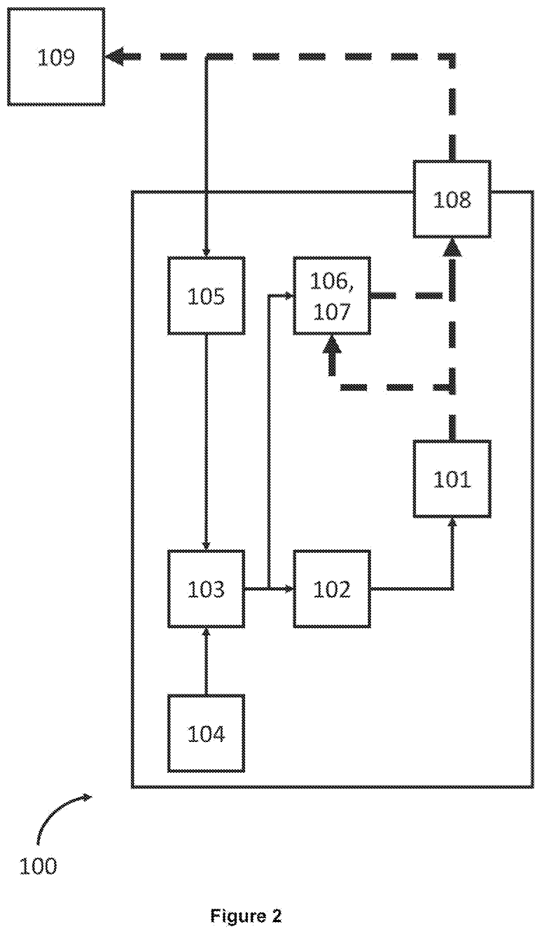

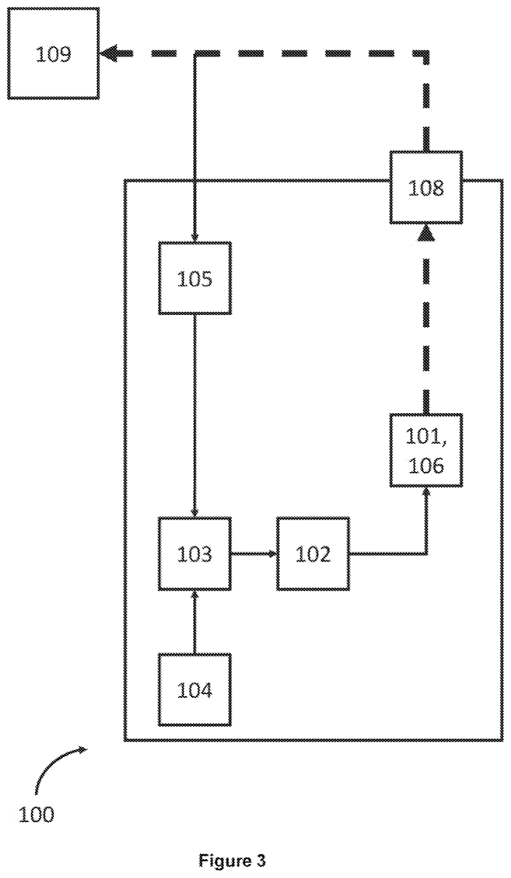

In some embodiments, alternatively or additionally, the fan 101 or the motor unit 102 and/or the valve 107 can be designed as the signal unit 106. For example, it can be provided that the device 100 comprises multiple signal units 106, which can generate various types of signals 204. It will be explained in more detail by way of example in FIGS. 2 and 3 how the fan 101 and/or the valve 107 can be used as signal units 106.

A further exemplary embodiment of the device 100 is schematically shown in FIG. 2. The exemplary device 100 includes at least one respiratory gas source in the form of a fan 101, which is driven by a motor unit 102 controllable by the control unit 103.

The device 100 furthermore includes a valve 107, which can switch between an insufflation 201 and an exsufflation 203, as also in the embodiment described in FIG. 1. The gas feed is switched here so that the suction side of the fan 101 is connected during insufflation 201 to an intake area for ambient air. For exsufflation 203, the valve is switched so that the suction side of the fan 101 is connected to the respiratory gas path to or from the patient 109 and thus generates a negative pressure to the patient 109.

The valve 107 is moreover configured as a signal unit 106 and can be activated by the control unit 103 to generate a pneumatic signal 204 in order to signal changing respiratory gas parameters. In particular, the signal 204 can signal switching 202 from insufflation 201 to exsufflation 203 or expiration 203a.

The signal 204 can be, for example, an adjustment of the pressure and/or flow during insufflation 201. It can also be provided that an oscillation can be generated as the signal 204 by deliberate switching of the valve 107.

For example, an adjustment of the pressure and/or flow to generate the signal 204 can thus take place. At a fixed and/or settable time before the switching, the pressure and/or the flow is increased or decreased in this case. A single brief reduction of the pressure can take place, for example, by briefly switching the valve 107, so that for a brief moment the suction side of the fan 101 is connected to the respiratory gas path from/to the patient 109 and thus briefly reduces the pressure there. In some embodiments, for example, for a longer time in which a lower pressure is to be used as the signal 204, a reduction of the speed of the fan 101 can be provided. Similarly, an increase of the pressure as the signal 204 can be achieved by an increase of the speed of the fan 101.

The sensor unit 105 and the user interface 104 are embodied by way of example as in the embodiments of FIG. 1.

FIG. 3 schematically shows an exemplary embodiment of the device 100 for respiratory therapy of a patient 109. The device 100 includes a respiratory gas source in the form of a fan 101 driven by a motor unit 102, which is also designed at the same time as a signal unit 106. The fan 101 is configured, for example, to provide an overpressure to the patient 109 during an insufflation 201. By suddenly switching to expiration 203a, for example sudden deceleration of the fan 101, an expiration can be forced and a cough can be triggered in the patient 109. A switch to the coughing phase thus takes place before the insufflation 201. In order to signal these changing respiratory gas parameters, among other things, a pneumatic signal can be generated by the signal unit 106.

To generate the signal 204, the motor 102 is activated by the control unit 103 so that the fan 101 can generate variations in the provided pressure during the insufflation 201. For example, it is provided that the generation of the signal 204 begins a time before the switching and then lasts for a period of time.

For example, an oscillatory signal 204 can be generated by a periodic reduction and increase 15 of the speed. In some embodiments, it can be provided that the entire insufflation 201 is overlaid with an oscillation, the signal 201 can then consist, for example, in the oscillation being modulated, thus changed in frequency and/or amplitude. It can also be provided that the signal 204 is generated by stopping the oscillation.

In some embodiments, it is provided that the signal 204 is a change of the pressure and/or flow. For example, to generate the signal 204, the speed of the fan 101 is increased, which results in an at least temporarily rising pressure and/or flow and signals changing respiratory gas parameters to the patient 109, in particular switching from insufflation 201 to expiration 203a. In some embodiments, it can be provided that the pressure and/or flow increase does not last over the entire period of time until switching, but only for a shorter period of time 25 before switching. Alternatively or additionally, it can be provided as the signal 204 that the signal 204 is an at least temporary reduction of the pressure. For example, the pressure is briefly reduced and then raised back to the plateau level of insufflation 201 before the switching takes place.

The sensor unit 105 and the user interface 104 are embodied, for example, as in the embodiments of FIG. 1.

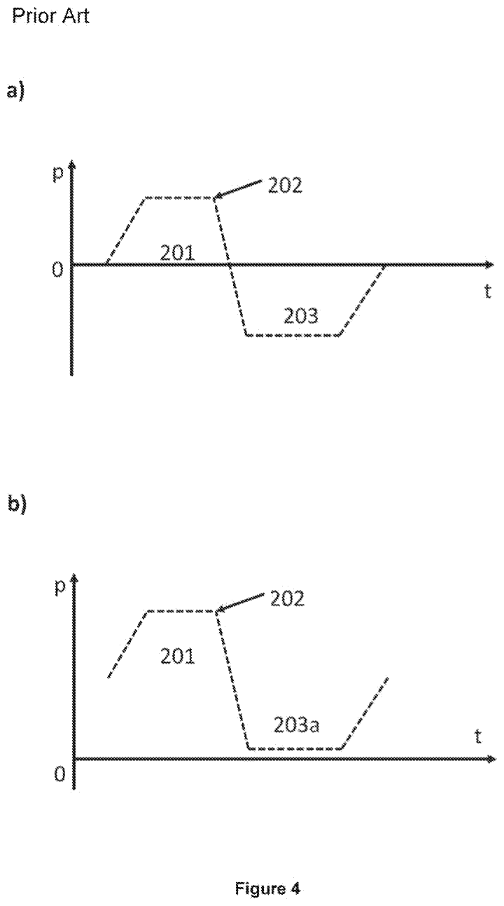

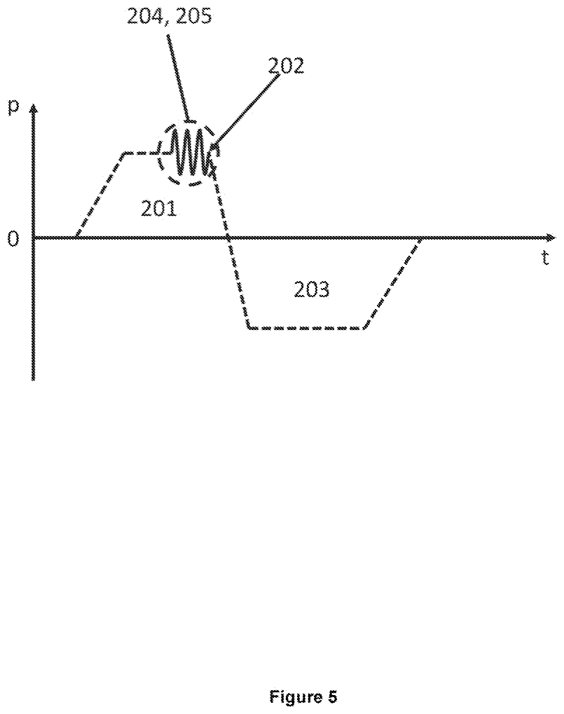

FIGS. 4 to 9 schematically show by way of example various pressure curves of an insufflation 201 and exsufflation 203 or expiration 203a. FIG. 4 a) describes by way of example a pressure curve of insufflation 201 and exsufflation 203 according to the prior art, FIG. 4 b) shows an expiration 203a instead of an exsufflation 203. In FIGS. 5 to 9, various signals 204 before the switching are shown by way of example on the basis of a pressure curve having insufflation 201 and exsufflation 203. The signals 204 can similarly also be transferred to embodiments in which exsufflation 203 does not take place after the switching, rather switching to an expiration 203a is provided. In the illustrated diagrams, the x axis designates the pressure p and the y axis designates the time t. The zero line shown relates here relative to the ambient pressure. A pressure p of zero corresponds here to the ambient pressure.

FIG. 4 schematically shows an exemplary pressure curve a) for an insufflation 201 with subsequent exsufflation 203 according to the prior art. At the beginning of insufflation 201, the pressure p increases until a plateau level is reached. This plateau is maintained for a time until insufflation 201 is ended and switching 202 to exsufflation 203 takes place. The pressure sinks rapidly due to the switching 202 to exsufflation 203 and forces the patient into an expiration and/or a cough. In exsufflation 203, a negative pressure (relative to the ambient pressure) is provided at the patient or a negative pressure is applied in the direction of the airways of the patient 109, by which the expiration or the cough is forced more strongly.

FIG. 4 also shows schematically an exemplary pressure curve b) for an insufflation 201 in which a switching 202 to expiration 203a takes place. In contrast to exsufflation 203, a negative pressure relative to the ambient pressure is not provided here, but rather the overpressure provided during insufflation 201 is strongly and suddenly reduced and approaches the ambient pressure in some embodiments. The patient is forced into an expiration 203a by the strong reduction of the pressure, possibly accompanied by a cough.

An exemplary embodiment of the signal 204 is schematically shown in FIG. 5. The signal 204 is shown, for example, in an embodiment having insufflation 201 and exsufflation 203, but can similarly also be transferred to embodiments in which insufflation 201 is followed by an expiration 203a, without a negative pressure being provided. After an increase, the pressure reaches a plateau level during insufflation 201, at which the pressure for the insufflation 201 is kept essentially constant and the patient 109 is ventilated using an overpressure. At a settable or fixed time before the switching 202, changing respiratory gas parameters, here, for example, the switching 202 from insufflation 201 to exsufflation 203, is signaled to the patient 109 by the signal 204. Changing respiratory gas parameters in this case are at least the pressure and/or the flow which change due to the switching 202. The signal 204 is generated, for example, for a settable or fixed period of time. This period of time can be, for example, between 0.1 seconds and 2 seconds, preferably between 0.5 seconds and 1.5 seconds. In some embodiments, the period of time of the signal 204 is preferably between 0.8 seconds and 1.2 seconds. The time before switching at which the generation of the signal 204 begins is, for example, between 0.1 seconds and 2 seconds, preferably between 0.5 seconds and 1.5 seconds, more preferably between 0.8 seconds and 1.2 seconds.

In the embodiment shown in FIG. 5, the signal 204 consists of an oscillation 205 of the pressure. The oscillation 205 can be generated, for example, by a periodic increase and decrease of the speed of a fan 101 and/or by the switching of an oscillation valve 110. In some embodiments, a valve 107 which switches over the suction side of the fan 101, can also generate an oscillation 205 of the pressure and thus a pneumatic signal 204.

The amplitude of the oscillation 205 is from about 0.2 hPa to about 50 hPa, preferably from about 0.5 hPa to about 30 hPa. In some embodiments, the amplitude is preferably from about 1 hPa to about 20 hPa. The amplitude refers here to the distance of the peaks from one another.

The frequency of the oscillation 205 is from about 1 Hz to about 40 Hz, preferably from about 2 Hz to about 30 Hz. In some embodiments, the frequency of the oscillation 205 is preferably from about 3 Hz to about 15 Hz.

In some embodiments, it can be provided that the frequency and/or amplitude of the oscillation 205 change by rising and/or change by falling during the generation of the signal. It can alternatively or additionally also be provided that the frequency and/or amplitude change dynamically. In some embodiments, it can be provided that the amplitude and/or frequency initially increases and then falls again. For example, amplitude and/or frequency can themselves isolate, thus periodically increase and decrease. In some embodiments, it can be provided that the frequency and/or amplitude of the oscillation 205 increase toward the point in time of the switching 202 and reach their maximum immediately before the switching 202.

It can be provided by way of example that the time before the switching 202, the period of time of the signal 204, the frequency and/or amplitude of the oscillation 205 are settable via the user interface 104. Additionally or alternatively, it can be provided that the time before the switching 202, the period of time of the signal 204, the frequency and/or amplitude of the oscillation 205 are fixed and/or can be selected from fixed values.

In some embodiments, it can be provided that the oscillation 205 used as the signal 204 is also used for measurement purposes, for example, to conclude various respiration parameters, such as the fill level of the lung of the patient 109 and/or secretion deposits, via respiratory gas signals.

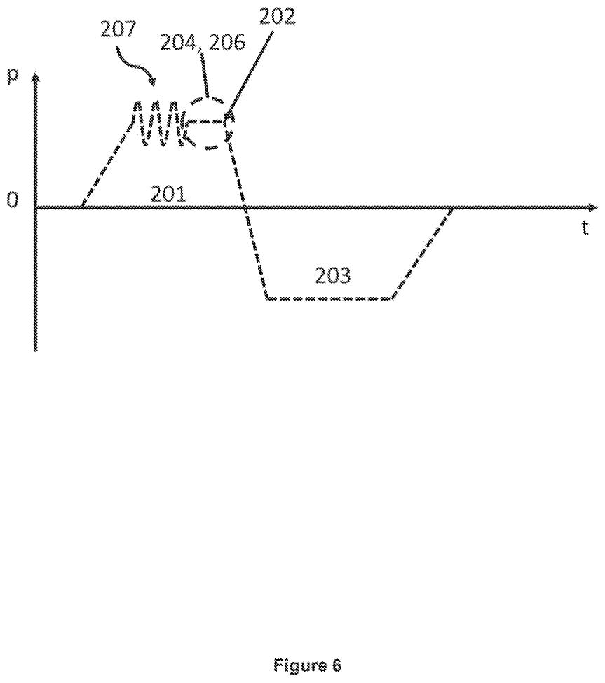

A further exemplary embodiment of the signal 204 for signaling changing respiratory parameters, in particular the switching 202 from insufflation 201 to exsufflation 203 or expiration 203a (not shown here) is schematically shown in FIG. 6.

For example, insufflation 201 is overlaid with an oscillation 207. The signal 204 consists in this case in switching off 206 the oscillation 207. At a fixed and/or settable time before the switching 202 from insufflation 201 to exsufflation 203, the oscillation 207 is switched off in order to signal to the patient 109 that after a period of time the switching 202 from insufflation 201 to exsufflation 203 will take place.

The period of time can be, for example, from about 0.1 seconds to about 2 seconds, preferably from about 0.5 seconds to about 1.5 seconds. In some embodiments, the period of time of the signal 204 is preferably from about 0.8 seconds to about 1.2 seconds. The time before the switching, at which the generation of the signal 204 begins, is, for example, from about 0.1 seconds to about 2 seconds, preferably from about 0.5 seconds to about 1.5 seconds, more preferably from about 0.8 seconds to about 1.2 seconds. In some embodiments, the period of time corresponds to the time before the switching.

In some embodiments, it can be provided that insufflation 201 takes place for a fixed and/or settable time. Alternatively or additionally, it can be provided that the time of insufflation 201 is dynamically adjusted, for example, on the basis of values picked up by the sensors 105. For example, a degree of filling of the lung of the patient 109 can be determined. If a specific degree of filling is reached, it can be provided that the switching 202 is signaled. In some embodiments, the oscillation 207 can be used to determine various respiration parameters of the patient 109, which are then evaluated, for example, as to whether switching 202 should take place.

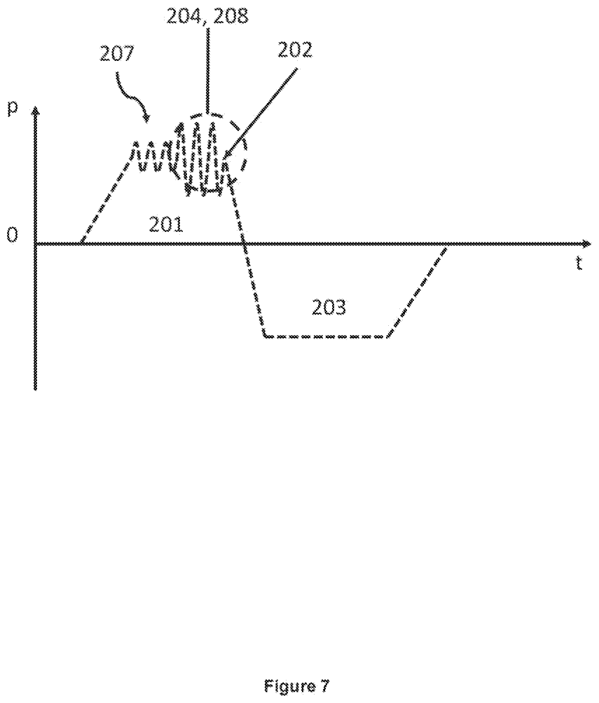

In some embodiments, in the case of an oscillation 207 during insufflation 201, a modulation 208 of the oscillation can also be provided to signal changing respiratory gas parameters, such as the switching 202 from insufflation 201 to exsufflation 203. Such an embodiment is shown by way of example in FIG. 7. At a time before the switching 202, a pneumatic signal 204 is generated for the patient 109 via a modulation 208 of the oscillation, which signals the imminent changing respiratory gas parameters. For example, the amplitude and/or frequency of the oscillation is increased or decreased.

The amplitude of the modulated oscillation 208 is from about 0.2 hPa to about 50 hPa, preferably from about 0.5 hPa to about 30 hPa. In some embodiments, the amplitude is preferably from about 1 hPa to about 20 hPa. The amplitude refers here to the distance of the peaks from one another.

The frequency of the modulated oscillation 208 is from about 1 Hz to about 40 Hz, preferably from about 2 Hz to about 30 Hz. In some embodiments, the frequency of the oscillation 205 is preferably from about 3 Hz to about 15 Hz.

The period of time of the signal 204 can be, for example, from about 0.1 seconds to about 2 seconds, preferably from about 0.5 seconds to about 1.5 seconds. In some embodiments, the period of time of the signal 204 is preferably from about 0.8 seconds to about 1.2 seconds. The time before the switching, at which the generation of the signal 204 begins, is, for example, from about 0.1 seconds to about 2 seconds, preferably from about 0.5 seconds to about 1.5 seconds, more preferably from about 0.8 seconds to about 1.2 seconds. In some embodiments, the period of time corresponds to the time before the switching.

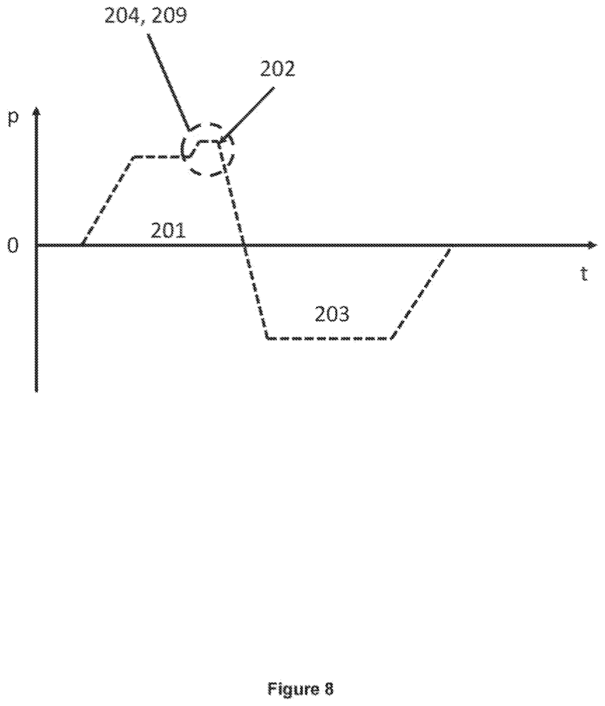

In some embodiments, the signal 204 consists in a temporary increase 209 of the pressure, as shown by way of example in FIG. 8. At a time before the switching 202, the plateau pressure of the insufflation 201 is raised further here in order to provide a then maximum pressure. This raised pressure 209 remains in existence, for example, for a period of time. The switching from insufflation 201 to exsufflation 203 then takes place from the raised pressure 209 of the signal 204 without returning to the plateau level. In some embodiments, it can alternatively or additionally be provided that the raised pressure 209 is not maintained until switching 202, but rather first sinks back to plateau level and the switching 202 to exsufflation 203 only takes place after a further period of time. Alternatively or additionally, it can be provided that the pressure increases continuously until the switching and reaches the maximum immediately before the switching 202.

The period of time of the signal 204 can be, for example, from about 0.1 seconds to about 2 seconds, preferably from about 0.5 seconds to about 1.5 seconds. In some embodiments, the period of time of the signal 204 is preferably from about 0.8 seconds to about 1.2 seconds.

The time before the switching, at which the generation of the signal 204 begins, is, for example, from about 0.1 seconds to about 2 seconds, preferably from about 0.5 seconds to about 1.5 seconds, more preferably from about 0.8 seconds to about 1.2 seconds. In some embodiments, the period of time corresponds to the time before the switching.

To generate the signal 204, the pressure can be temporarily raised, for example, in a range of from about 0.2 hPa to about 8 hPa. The pressure is preferably temporarily raised in a range from about 0.5 hPa to about 5 hPa. The raising 209 of the pressure can be generated, for example, via an increased speed of the fan 101.

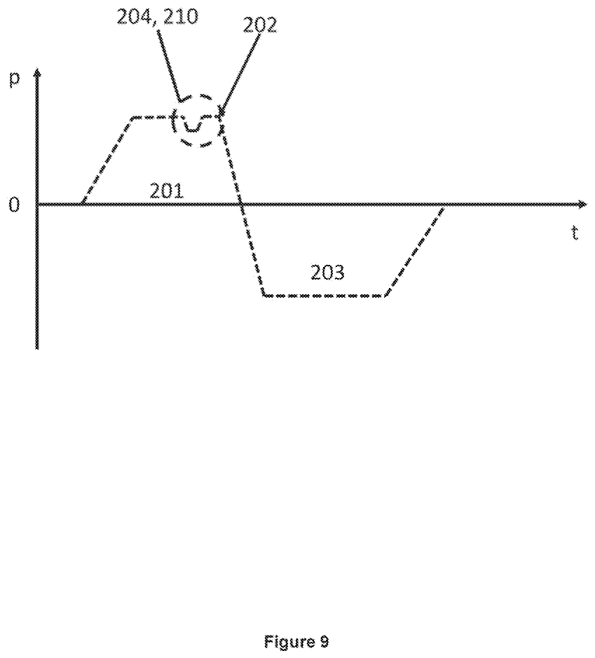

In some embodiments, the signal 204 consists in a temporary reduction 210 of the pressure, as shown by way of example in FIG. 9. At a time before the switching 202, the plateau pressure of the insufflation 201 is at least temporarily lowered. This lowered pressure 210 remains in existence for a period of time, for example. The switching from insufflation 201 to exsufflation 203 then takes place from the lowered pressure 210 of the signal 204 without returning to the plateau level. In some embodiments, it can alternatively or additionally be provided that the lowered pressure 210 is not maintained until the switching 202, but rather is first raised back to plateau level and the switching 202 to exsufflation 203 only takes place after a further period of time. Alternatively or additionally, it can be provided that the pressure decreases continuously until the switching and switching takes place from there directly to exsufflation 203.

The period of time of the signal 204 can be, for example, from about 0.1 seconds to about 2 seconds, preferably from about 0.5 seconds to about 1.5 seconds. In some embodiments, the period of time of the signal 204 is preferably from about 0.8 seconds to about 1.2 seconds.

The time before the switching, at which the generation of the signal 204 begins, is, for example, from about 0.1 seconds to about 2 seconds, preferably from about 0.5 seconds to about 1.5 seconds, more preferably from about 0.8 seconds to about 1.2 seconds. In some embodiments, the period of time corresponds to the time before the switching.

To generate the signal 204, the pressure can be temporarily lowered, for example, in a range of from about 0.2 hPa to about 8 hPa. The pressure is preferably temporarily lowered in a range from about 0.5 hPa to about 5 hPa. The lowering 210 of the pressure can be generated, for example, via a decreased speed of the fan 101.

To sum up, the present invention provides:
1. A device for respiratory therapy of a patient, wherein the device comprises a respiratory gas source for specifying different respiratory gas parameters, comprising at least one control unit, and a signal unit for outputting at least one signal, the at least one signal being used for signaling changing respiratory gas parameters and being sensorially perceptible by the patient.
2. The device of item 1, wherein the signal unit emits the signal to the patient before a change of a respiratory gas parameter.
3. The device of at least one of the preceding items, wherein the at least one signal is transmitted to an airway of the patient pneumatically—thus via the air path (device, hose system; patient interface)—and includes a modulation of a specified respiratory gas with respect to pressure and/or flow and/or volume.
4. The device of at least one of the preceding items, wherein the at least one signal is sensorially perceptible by the patient via her mechanoreceptors and/or cold receptors and/or smell receptors and/or taste receptors.
5. The device of at least one of the preceding items, wherein the signal unit is configured for generating pneumatic signals and includes, for example, a valve and/or a fan.
6. The device of at least one of the preceding items, wherein the signal unit is controlled by the control unit.
7. The device of at least one of the preceding items, wherein the changing respiratory gas parameter is a respiratory gas pressure provided by the device.
8. The device of least one of the preceding items, wherein the respiratory gas parameter is changed upon switching from insufflation to coughing phase (expiration and/or exsufflation).
9. The device of at least one of the preceding items, wherein the changing respiratory gas parameter is a positive respiratory gas pressure, which is dissipated following an insufflation phase for a coughing phase (expiration) or is switched to negative pressure (exsufflation).
10. The device of at least one of the preceding items, wherein the device carries out an insufflation with overlaid oscillation, the at least one signal being an oscillation which is changed before switching to a coughing phase (exsufflation and/or expiration).
11. The device of at least one of the preceding items, wherein the device carries out an insufflation without oscillation, the at least one signal being a specific modulation of the respiratory gas with respect to pressure and/or flow and/or volume.
12. The device of at least one of the preceding items, wherein the at least one signal includes a modulation of pressure and/or flow (pneumatic signal).
13. The device of at least one of the preceding items, wherein the at least one signal is a modulated-on oscillation having a fixed or changing frequency and/or amplitude.
14. The device of item 13, wherein the oscillation has a frequency from about 1 to about 40 Hz, preferably a frequency from about 2 to about 30 Hz, and an amplitude of the oscillation (peak to peak) is from about 0.2 to about 50 hPa, preferably from about 0.5 to about 30 hPa.
15. The device of at least one of the preceding items, wherein the at least one signal is a brief positive or negative pressure and/or flow pulse on an air column.
16. The device of at least one of the preceding items, wherein the at least one signal changes between the beginning of the at least one signal and a moment of switching.
17. The device of at least one of the preceding items, wherein the device further comprises a generator for generating a detector signal and a sensor for determining a change of the detector signal, the detector signal being suitable for detecting changes of a patency of the airways (closure of glottis) and/or an at least advanced or complete filling of the lung and the sensor determining this change of the detector signal.
18. The device of item 17, wherein automatic switching to the coughing phase takes place when, based on the detector signal, a change of the patency of the airways (closure of the glottis) or an at least advanced or complete filling of the lung is determined.
19. The device of at least one of items 17 and 18, wherein the automatic switching is based on an oscillatory pressure, flow, and/or volume signal.
20. The device of at least one of items 17 to 19, wherein the switching also takes place before expiration of a stored or set period of time if a glottis closure is established metrologically by a detector signal.
21. The device of at least one of items 17 to 20, wherein an oscillation takes place during insufflation, the oscillation being simultaneously used as basis for the detector signal and switching takes place when, based on the detector signal, a closure of the glottis and/or an at least substantially completely filled lung can be concluded before expiration of the set insufflation time.
22. The device of at least one of items 17 to 21, wherein the oscillation as basis for the detector signal for the glottis closure is added in the course of the insufflation, the insufflation being ended by a detected glottis closure or with expiration of the set maximum insufflation time.
23. The device of at least one of the preceding items, wherein the pneumatic signal is generated for a fixed and/or settable period of time and/or wherein the pneumatic signal is generated from a fixed and/or settable time before switching to the coughing phase.
24. A system for respiratory therapy of a patient, wherein the system comprises a device according to at least one of the preceding items, and further comprises a patient interface and a respiratory gas hose.

What is claimed is:

1. A device for respiratory therapy of a patient, wherein the device comprises a respiratory gas source for specifying different respiratory gas parameters, comprising at least one control unit, and a signal unit for outputting at least one signal, the at least one signal being used for signaling changing respiratory gas parameters and being sensorially perceptible by the patient.

2. The device of claim 1, wherein the signal unit emits the signal to the patient before a change of a respiratory gas parameter.

3. The device of claim 1, wherein the at least one signal is transmitted to an airway of the patient pneumatically and includes a modulation of a specified respiratory gas with respect to pressure and/or flow and/or volume.

4. The device of claim 1, wherein the at least one signal is sensorially perceptible by the patient via her mechanoreceptors and/or cold receptors and/or smell receptors and/or taste receptors.

5. The device of claim 1, wherein the signal unit is configured for generating pneumatic signals.

6. The device of claim 1, wherein the signal unit is controlled by the control unit.

7. The device of claim 1, wherein the changing respiratory gas parameter is a respiratory gas pressure provided by the device.

8. The device of claim 1, wherein the respiratory gas parameter is changed upon switching from insufflation to coughing phase.

9. The device of claim 1, wherein the changing respiratory gas parameter is a positive respiratory gas pressure, which is dissipated following an insufflation phase for a coughing phase or is switched to negative pressure.

10. The device of claim 1, wherein the device carries out an insufflation with overlaid oscillation, the at least one signal being an oscillation which is changed before switching to a coughing phase.

11. The device of claim 1, wherein the device carries out an insufflation without oscillation, the at least one signal being a specific modulation of the respiratory gas with respect to pressure and/or flow and/or volume.

12. The device of claim 1, wherein the at least one signal includes a modulation of pressure and/or flow.

13. The device of claim 1, wherein the at least one signal is a modulated-on oscillation having a fixed or changing frequency and/or amplitude.

14. The device of claim 13, wherein the oscillation has a frequency from 1 to 40 Hz, and an amplitude of the oscillation is from 0.2 to 50 hPa.

15. The device of claim 1, wherein the at least one signal is a brief positive or negative pressure and/or flow pulse on an air column.

16. The device of claim 1, wherein the at least one signal changes between a beginning of the at least one signal and a moment of switching.

17. The device of claim 1, wherein the device further comprises a generator for generating a detector signal and a sensor for determining a change of the detector signal, the detector signal being suitable for detecting changes of a patency of airways and/or an at least advanced or complete filling of a lung and the sensor determining this change of the detector signal.

18. The device of claim 17, wherein automatic switching to a coughing phase takes place when, based on the detector signal, a change of the patency of the airways or an at least advanced or complete filling of the lung is determined.

19. The device of claim 18, wherein the automatic switching is based on an oscillatory pressure, flow, and/or volume signal.

20. A system for respiratory therapy of a patient, wherein the system comprises the device of claim 1, and further comprises a patient interface and a respiratory gas hose.
